(19) United States
(12) Patent Application Publication
(10) Pub. No.: US 2008/0207700 A1
(43) Pub. Date: Aug. 28, 2008

(54) Amino Acid Salts of Rosiglitazone

(76) Inventors: Udo Werz, Achstetten-Stetten (DE); Gerhard Maas, Senden (DE); Heinrich Stahl, Freiburg (DE)

Correspondence Address: SMITH PATENT CONSULTING CONSULTING, LLC, 3309 DUKE STREET, ALEXANDRIA, VA, 22314, US

(21) Appl. No.: 11/996,395

(22) PCT Filed: Jul. 20, 2006
(86) PCT No.: PCT/EP06/07171

§ 371 Date: Jan. 22, 2008

(30) Foreign Application Priority Data
Jul. 22, 2005 (DE) 10 2005 034 406.2

(51) Int. Cl. A61K 31/4436; C07D 417/06; A61P 3/10

(52) U.S. Cl. 514/342; 546/269.7

(57) ABSTRACT

The invention relates to novel amino acid salts of the racemic or an enantiomeric or tautomeric form of rosiglitazone and to the solvates of said salts.

The present invention relates to new salts of rosiglitazone, namely amino acid salts of rosiglitazone and the solvates thereof, to pharmaceutical preparations containing such salts or solvates, to the use thereof for treating certain diseases, and to processes for producing such salts. In particular, the invention relates to the cholinate, lysinate and arginate of the racemic or an enantiomeric or tautomeric form of rosiglitazone, the cholinate also being preferred because of its good water solubility.

Rosiglitazone is the INN designation for 5-(4-/2-(N-methyl-N-(2-pyridyl)amino)ethoxy/benzyl)-2,4-thiazolidinedione and is described in detail in EP-B 0 306 228 B1. It is suited for the treatment and prevention of hyperglycemia, in particular type II diabetes, hyperlipemia, high blood pressure, cardiovascular diseases and certain eating disorders. The maleate salt is said to be better soluble than the free base, have good stability and on account of its improved selectivity be usable in particular for type II diabetes. It is described in EP 0 658 161 B1. WO94/05659 additionally discloses the tartrate salt. WO02/12232 discloses the DL tartrate which is supposed to differ from the D tartrate and the L tartrate and have advantageous properties. The hydrochloride salt of rosiglitazone is the subject matter of WO02/20519 and the phosphate salt is disclosed in WO05/023803. It shall have a high water solubility which is, however, not yet quite satisfactory. Therefore, there is a need for new salts of rosiglitazone broadening the possible uses thereof. The new salts shall have in particular a good solubility, especially under physiological conditions.

A criterion for the possible applications of new rosiglitazone salts is that substances which might have disadvantageous or even harmful properties are not taken into the body by the pharmaceutically non-active anion which is said to change certain secondary properties of the pharmaceutically active base. Thus, the anion shall not be foreign to the body and, if possible, shall be a substance which is present in the body anyway or whose supply might even be advantageous.

It has now been found that the amino acids are beneficial to the salt formation of rosiglitazone. The amino acids are partially even essential constituents of the body, i.e. no substances foreign to the body, and their supply is often actually desired. Thus, the amino acid salts according to the invention are well tolerated and show low toxicity. They are also well soluble in water. The water solubility depends on the pH. With pH 9.0 the rosiglitazone cholinate has a water solubility of 20.0 mg/ml, the rosiglitazone lysinate has one of 9.4 mg/ml, while that of the rosiglitazone maleate is 5.9 mg/ml and that of the rosiglitazone phosphate is only 2.4 mg/ml. With pH 6 the water solubility of the rosiglitazone cholinate is 11.7 mg/ml and thus over a hundred times greater than that of the rosiglitazone maleate (<0.1 mg/ml). Surprisingly, the salts according to the invention are virtually not hygroscopic and show excellent stability. In so far as the invention is described here for rosiglitazone, the invention applies likewise to the enantiomers and to tautomeric forms of rosiglitazone.

The salt of rosiglitazone with choline is particularly preferred for the time being.

Choline is an important component in numerous metabolic functions and is used as a therapeutic. In addition, it is a constituent of multivitamin preparations and is contained in many foodstuffs. Taken in common amounts it is virtually non-toxic and therefore well compatible.

Lysine is an essential amino acid and is present in almost all proteins. Its pharmaceutical compatibility has been tested many times over. Lysine is used as a food additive in particular for dietetic foodstuffs.

Arginine is a non-essential amino acid which also occurs in almost all proteins. It is used as both food additive and constituent of therapeutics.

The amino acid salts can easily be produced by dissolving the rosiglitazone base in boiling ethanol or methanol and adding the amino acid as a solid or in solution in warm water. The rosiglitazone cholinate can also be obtained appropriately by providing a suspension of rosiglitazone in dried ethanol, mixing it with a choline solution and precipitating the salt with ethyl acetate and diethyl ether. The product precipitates as crystals and is preferably filtered off at 0° C.

The salts according to the invention can be formulated in generally known manner into pharmaceutical preparations for mammals, preferably humans. The pharmaceutical preparations contain the salts according to the invention in admixture with a pharmaceutical organic or inorganic carrier which is suited for enteral or parenteral administrations. The oral administration of the salts according to the invention via tablets, capsules, powders or in liquid form, such as suspensions, in solution as an emulsion or as syrup is particularly preferred.

When tablets are formulated, common drug carriers are used, such as sodium citrate, lactose, microcrystalline cellulose and starch, lubricants, such as anhydrous silica, hydrogenated castor oil, magnesium stearate, sodium lauryl sulfate and talcum, as well as binders, such as starch paste, glucose, lactose, gum Arabic, mannitol, magnesium trisilicate and talcum. When the salts according to the invention are administered via liquids, common liquid carriers can be used.

A formulation for injections and infusions as known in the art and described in relevant standard works is also preferred.

The salts according to the invention can also be formulated in generally known manner as depot formulations or into medicaments having a delayed or sustained release.

### EXAMPLES

### Example 1

3.5 g rosiglitazone were dissolved in 15 ml THF at a bath temperature of 50° C. 2.65 g 45% methanolic choline hydroxide solution were added to the solution. Having stirred for 5 minutes, 75 ml ethyl acetate were gradually added while stirring. Seed crystals were added to the slightly turbid solution and the bath was removed. The batch was allowed to stand at room temperature overnight. The product precipitated as fine white needles. The crystals were isolated by vacuum filtration, washed with 10 ml of a mixture of THF/ethyl acetate 1:3 and dried in vacuo for 24 h.

Yield: 3 g

Melting point: 101.5-103.8° C.

¹H spectrum: composition choline/rosiglitazone 1:1, virtually no solvent visible

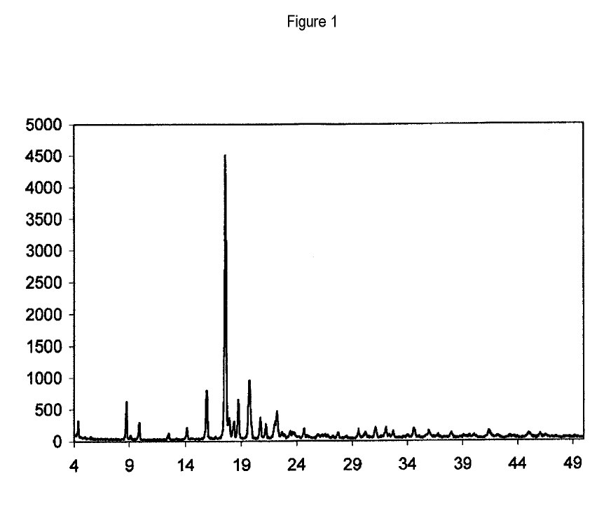

A powder X-ray spectrum of the product was taken which is shown in FIG. 1. The 2 θ value is plotted on the x-axis, and the intensity is plotted on the y-axis. The resulting polymorphous form is characterized by the main peaks at 2 θ of 8.76, 15.90, 17.59, 18.75, 19.73 and 22.24, in particular by the following peak list:

### Powder X-ray Diffraction

| Peak position 2 θ (°) | Peak intensity |
|---|---|
| 8.76 | 571.3 |
| 9.89 | 283.3 |
| 12.49 | 119.9 |
| 14.12 | 206.7 |
| 15.90 | 804.3 |
| 17.59 | 4529.8 |
| 17.92 | 319.2 |
| 18.34 | 290.5 |
| 18.75 | 669.7 |
| 19.73 | 952.5 |
| 20.72 | 369.8 |
| 21.22 | 265.5 |
| 22.06 | 309.9 |
| 22.24 | 463.8 |
| 23.41 | 161.8 |
| 24.68 | 189.6 |
| 29.56 | 184.1 |
| 31.07 | 187.3 |
| 32.01 | 201.0 |
| 34.57 | 184.4 |

The measurement was made as usual with standard methods at room temperature and normal pressure. 0.2 can be specified as an error range for each 2 θ value.

### Example 2

5 g rosiglitazone were supplied in 25 ml dry ethanol as a suspension, 3.87 g choline solution were added at room temperature, the mixture was stirred for 10 min and finally filtrated. 100 ml ethyl acetate and 100 ml diethyl ether were added to the filtrate which was then placed in a refrigerator at 5° C. overnight. The product precipitated as fine white needles. The crystals were isolated by vacuum filtration and washed with 10 ml diethyl ether. The product was dried at room temperature at 20 mbar for 5 days.

Yield: 4.5 g (70%)

### Example 3

4 g rosiglitazone were dissolved in 130 ml dry ethanol at boiling heat. 1.642 g lysine were dissolved in 5 ml warm water and added to the hot rosiglitazone solution. A clear solution formed which was heated to boiling for 5 min. The heating bath was removed, and the batch was allowed to stand at room temperature. After 3 h of standing at room temperature, it was placed in a refrigerator at 5° C. overnight. The product was isolated by vacuum filtration, washed with 20 ml ethanol and dried in a vacuum at 50° C. for several days.

Yield: 5.4 g

### Example 4

1 g rosiglitazone and 411 mg lysine were dissolved in 10 ml boiling methanol (dried) and added to 12 ml isopropanol at boiling heat. After about 5 min, the heating bath was removed and the batch was allowed to stand at room temperature overnight. The product was isolated by vacuum filtration, washed with isopropanol and diethyl ether, and then dried in a vacuum at 50° C. for 16 h.

¹H spectrum: composition lysine/rosiglitazone 1:1, about 6% by mole isopropanol.

### Example 5

1.05 g rosiglitazone and 426 mg lysine were dissolved in 10 ml boiling methanol (dried) and added to 18 ml ethyl acetate at boiling heat. After about 5 min, the heating bath was removed and the batch was allowed to stand at room temperature overnight. The product was isolated by vacuum filtration, washed with ethyl acetate and dried in the vacuum at 50° C. for 16 h.

Yield: 1 g

¹H spectrum: composition lysine/rosiglitazone 1:1, about 8% by mole ethanol

### Example 6

2 g rosiglitazone and 976 mg arginine were heated to boiling in 70 ml ethanol for half an hour. The resulting solution was allowed to stand at room temperature overnight. The product was isolated by vacuum filtration, washed with ethanol and dried in a vacuum at 50° C. for 2 days.

Yield: 2.4 g

### Example 7

The solubility of the rosiglitazone salts produced in Examples 1 and 3 was determined and compared with the solubility of the maleate salt and the free base.

The following buffer solutions were used:

| | |
|---|---|
| pH 1.5 | 2% phosphoric acid |
| pH 3.0 | 188 mg KH2PO4 dissolved in 200 ml aqua purificata and |
| | adjusted with 1.0n HCl |
| pH 8.9 | 188 mg KH2PO4 dissolved in 200 ml aqua purificata and |
| | adjusted with 1.0n NaOH |
| pH 12.0 | 250 mg K2HO4 dissolved in 200 ml aqua purificata and adjusted |
| | with 1.0n HCl |

About 100 mg of the substance for investigation in 10 ml (with good solubility such as e.g. pH 11.8 correspondingly less) of the corresponding above mentioned buffer solutions were added in each case and raised to the corresponding pH by means of 1.0 n HCl or 1.0 n NaOH. Thereafter, the suspensions were treated in an ultrasonic bath for 1 minute, the pH was checked and, where necessary, readjusted to the value given respectively below. Then, the solutions were 0.45 μm filtrated.

The absorption of thus produced solutions was measured at 345 nm. The test solutions were uniformly adjusted to a pH of 1.5 for the measurement with phosphoric acid. The free base served as a calibrating substance.

The result is summarized in Table 1. It discloses that, above all in the physiological pH range, the amino acid salts have a solubility markedly better than that of the base and the maleate salt, in particular the cholinate shows an over 100 times greater solubility at pH 6.5.

TABLE 1
Solubility of the amino acid salts of rosiglitazone as
compared to the maleate salt and the free base.

| Solubility | | | | |
|---|---|---|---|---|
| at | Cholinate | Lysinate | Maleate | Base |
| pH 4.6 | 1.1 mg/ml | 1.2 mg/ml | 0.6 mg/ml | 0.2 mg/ml |
| pH 6.5 | 11.7 mg/ml | 0.2 mg/ml | <0.1 mg/ml | 0.1 mg/ml |
| pH 9.0 | 20.0 mg/ml | 9.4 mg/ml | 5.9 mg/ml | 5.9 mg/ml |
| pH 11.8 | >50 mg/ml | >50 mg/ml | 19.2 mg/ml | >50 mg/ml |

1. Amino acid salts of a racemic, enantiomeric, or tautomeric form of rosiglitazone and solvates of said salts.

2. The amino acid salt or solvate according to claim 1, wherein the rosiglitazone is rosiglitazone cholinate.

3. The amino acid salt or solvate according to claim 1, wherein the rosiglitazone is rosiglitazone lysinate.

4. The amino acid salt or solvate according to claim 1, wherein the rosiglitazone is rosiglitazone arginate.

5. A polymorphous form of rosiglitazone cholinate, characterized by a powder X-ray diffractogram with 2 θ values at 8.76, 15.90, 17.59, 18.75, 19.73 and 22.24.

6. A medicament comprising an amino acid salt or solvate according to claim 1 and optionally including one or more pharmaceutically compatible carriers and/or excipients.

7. A method for treating or preventing a condition selected from the group consisting of hyperglycemia, hyperlipemia, high blood pressure, cardiovascular disease and eating disorders, said method comprising the step of administering to a subject in need thereof an effective amount of an amino acid salt or solvate according to claim 1.

8. A process for producing an amino acid salt or solvate according to claim 1, characterized by the step of (a) reacting the racemic, enantiomeric, or tautomeric form of rosiglitazone with an amino acid or (b) converting a salt of rosiglitazone with an amino acid into another salt.

9. The method of claim 7, wherein said condition is type II diabetes.
